(19) United States
(12) Patent Application Publication
(10) Pub. No.: US 2026/0232246 A1
(43) Pub. Date: Aug. 13, 2026

(54) BIOSIGNAL MEASUREMENT SYSTEM

(71) Applicant: NTT, Inc, Tokyo (JP)

(72) Inventors: Kento Watanabe, Tokyo (JP); Kenichi Matsunaga, Tokyo (JP)

(21) Appl. No.: 18/872,637

(22) PCT Filed: Jun. 9, 2022
(86) PCT No.: PCT/JP2022/023296

§ 371 Date: Dec. 6, 2024

(51) Int. Cl. A61B 5/308; A61B 5/28

(52) U.S. Cl. CPC A61B 5/308; A61B 5/28

(57) ABSTRACT

An embodiment is a biosignal measurement system including a plurality of electrode devices, each including an electrode, an amplifier circuit configured to amplify a biopotential measured via the electrode, a quantization circuit configured to convert the biopotential amplified by the amplifier into digital data to generate biopotential information, a transmitter configured to transmit the biopotential information, and a receiver configured to receive reference potential information of the amplifier, and a biosignal generation device including a wireless receiver configured to receive the biopotential information transmitted from the plurality of electrode devices, an arithmetic circuit configured to generate a biosignal waveform and the reference potential information using the biopotential information in at least two of the plurality of electrode devices, and a transmitter configured to transmit the reference potential information to the plurality of electrode devices, where the amplifier of each of the plurality of electrode devices is configured to amplify the biopotential using reference potential information received from the biosignal generation device.

### CROSS-REFERENCE TO RELATED APPLICATIONS

This application is a national phase entry of PCT Application No. PCT/JP2022/023296, filed on Jun. 9, 2022, which application is hereby incorporated herein by reference.

### TECHNICAL FIELD

The present disclosure relates to a biosignal measurement system for measuring a biosignal including an electrocardiographic waveform.

### BACKGROUND

In recent years, as one of methods for health management of individuals, biosignals such as electrocardiographic waveforms are recorded over a long period of time, and characteristics and changes of the waveforms are analyzed to find out the degree of activity of autonomic nerves and signs of heart disease at an early stage. As a method of acquiring a biosignal such as an electrocardiographic waveform over a long period of time, a wearable electrode in which a bioelectrode is attached to clothing has been proposed (see, for example, Non Patent Literature 1).

In the electrocardiographic waveform, which is one of biosignals, it is necessary to measure a potential difference between electrodes disposed on both left and right sides across the heart of the body. As illustrated in FIG. 8, a wearable electrode 100 of Non Patent Literature 1 includes a biopotential measuring device 400 attached to a central portion of a body, and electrodes (200, 300) which are brought into contact with left and right waist portions by wiring on compression wear.

### CITATION LIST

### Non Patent Literature

Non Patent Literature 1: Nahoko Kasai, Takayuki Ogasawara, Hiroshi Nakashima, and Shingo Tsukada, “Development of Functional Textile “hitoe”: Wearable Electrodes for Monitoring Human Vital Signals”, Communication Society Magazine, 2017-2018, Vol. 11, No. 1, pp. 17-23, The Institute of Electronics, Information and Communication Engineers, publication date: 2017 Jun. 1

### SUMMARY

### Technical Problem

When the bioelectrode is worn on the body by wearing, time and effort for wearing may cause a sense of repulsion to the wearer, and discomfort may be given to the wearer by a sense of pressure due to wearing. As an attachment position other than the body, for example, in a case where the electrodes are attached to the limbs, since the wiring linking the left and right electrodes forms a loop like handcuffs, there is a problem that the movement of the body of the wearer is constrained and a strong constraint exists.

An object of embodiments of the present invention is to solve the above problem, and an object is to provide a biosignal measurement system capable of naturally measuring a biosignal by eliminating discomfort of a wearer and constraint on the body at the time of wearing an electrode device.

### Solution to Problem

In order to solve the above problem, a biosignal measurement system of embodiments of the present invention includes: a plurality of electrode devices including an electrode that measures a biopotential, an amplifier circuit that amplifies the measured biopotential, a quantization circuit that converts the amplified biopotential into digital data to generate biopotential information, a wireless transmitter that transmits the biopotential information, a wireless receiver that receives reference potential information of the amplifier circuit, and a power supply that supplies power to the amplifier circuit, the quantization circuit, the wireless transmitter, and the wireless receiver; and a biosignal generation device including a wireless receiver that receives the biopotential information transmitted from the wireless transmitter of the electrode device, an arithmetic circuit that generates a biosignal waveform and the reference potential information using the biopotential information in at least two electrode devices of the plurality of electrode devices, and a wireless transmitter that transmits the generated reference potential information to the electrode device, in which the amplifier circuit of the electrode device amplifies the biopotential using reference potential information received from the biosignal generation device.

### Advantageous Effects of Embodiments of the Invention

According to embodiments of the present invention, it is possible to provide a biosignal measurement system capable of naturally measuring a biosignal by eliminating discomfort of a wearer and constraint on the body at the time of wearing an electrode device.

### BRIEF DESCRIPTION OF THE DRAWINGS

FIG. 1 is a diagram illustrating a configuration example of a biosignal measurement system according to a first embodiment of the present invention.

FIG. 2 is a diagram illustrating a conceptual diagram of a biosignal measurement system according to an embodiment of the present invention.

FIG. 3 is an example of a measurement circuit used in a conventional biosignal measurement system.

FIG. 4 is a diagram illustrating a configuration example of a biosignal measurement system according to a second embodiment of the present invention.

FIG. 5 is a diagram illustrating a configuration example of a biosignal measurement system according to a third embodiment of the present invention.

FIG. 6 is a diagram illustrating an example of an operation flow in a reference potential correction circuit according to a third embodiment of the present invention.

FIG. 7 is a diagram illustrating a configuration example of a reference potential correction circuit according to a fourth embodiment of the present invention.

FIG. 8 is a configuration example of a conventional biosignal measurement system.

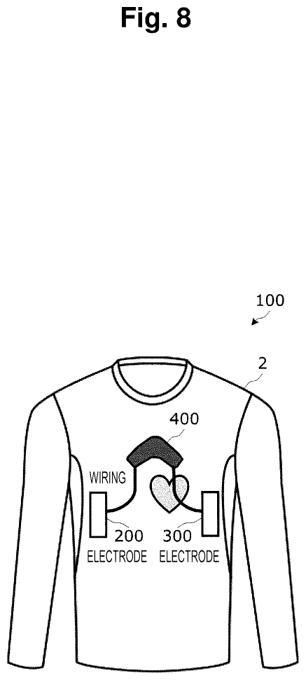

### DETAILED DESCRIPTION OF ILLUSTRATIVE EMBODIMENTS

Hereinafter, modes for carrying out embodiments of the present invention will be described with reference to the drawings. The contents of the present invention are not limited by the embodiments described below.

### First Embodiment

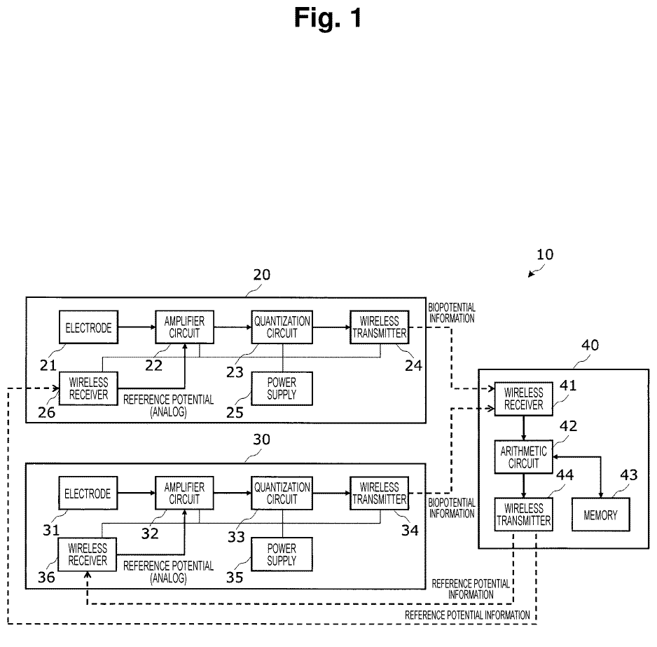

FIG. 1 is a diagram illustrating a configuration example of a biosignal measurement system according to a first embodiment of the present invention. A biosignal measurement system 10 of the present embodiment includes a plurality of electrode devices (20, 30) that measure a biopotential and a biosignal generation device 40 that generates a biosignal waveform using biopotential information in the plurality of electrode devices (20, 30).

The electrode devices (20, 30) include electrodes (21, 31) that measure a biopotential, amplifier circuits (22, 32) that amplify the measured biopotential, quantization circuits (23, 33) that convert the amplified biopotential into digital data to generate biopotential information, wireless transmitters (24, 34) that transmit the biopotential information, wireless receivers (26, 36) that receive reference potential information from the biosignal generation device 40, and power supplies (25, 35) that supply power to the amplifier circuits (22, 32), the quantization circuits (23, 33), the wireless transmitters (24, 34), and the wireless receivers (26, 36). The amplifier circuits (22, 32) amplify the biopotential using the reference potential information received from the biosignal generation device 40.

The biosignal generation device 40 includes a wireless receiver 41 that receives biopotential information transmitted from the wireless transmitters (24, 34) of the electrode devices (20, 30), an arithmetic circuit 42 that generates a biosignal waveform and biopotential information of the amplifier circuits (22, 32) using the biopotential information in at least two electrode devices of the plurality of electrode devices (20, 30), and a wireless transmitter 44 that transmits the generated reference potential information to the electrode devices (20, 30).

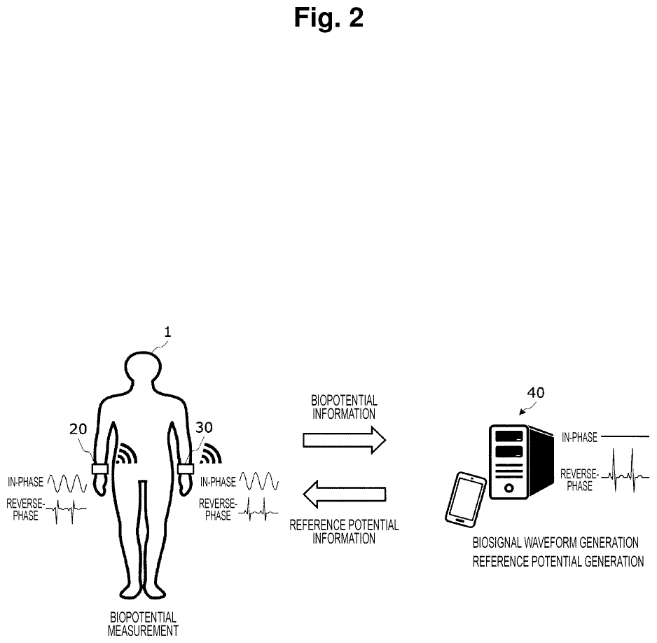

FIG. 2 illustrates a conceptual diagram of the biosignal measurement system 10 according to the present embodiment. For example, when an electrocardiogram is generated as a biosignal, it is necessary to dispose a plurality of electrode devices (20, 30) at positions sandwiching the heart. As an attachment form of the electrode devices (20, 30) having a good sense of use for a wearer 1, for example, it is conceivable to attach the electrode devices (20, 30) to the limbs such as hands and feet. By adopting such an attachment form of the electrode devices (20, 30), it is possible to greatly reduce a sense of pressure or discomfort due to wearing of wear or the like.

In each electrode device (20, 30), an in-phase component appearing as a noise component and a reverse-phase component appearing as a biopotential are measured. There is a problem that the signal of the biopotential that can be measured by the electrodes (21, 31) of the electrode devices (20, 30) is weak and the SN ratio is extremely poor.

In the biosignal measurement system 10 according to the present embodiment, signals of a plurality of measured biopotentials are transmitted to the biosignal generation device 40 by using wireless communication, and a difference operation of the signals of the biopotentials is performed by the biosignal generation device 40. By performing the difference operation, the in-phase component appearing as the noise component is removed to generate the biosignal, so that the SN ratio can be improved.

The biosignal measurement system of the present embodiment is configured such that the transmission of the information of the biopotential from the plurality of electrode devices for measuring the biopotential to the biosignal generation device for generating the biosignal waveform is performed by using the wireless communication, and therefore, it is possible to provide the biosignal measurement system capable of naturally measuring a biosignal by eliminating discomfort of the wearer and constraint on the body due to physical wiring at the time of wearing the electrode devices.

In FIG. 2, the case where the electrocardiogram, which is one of the biosignals, is measured has been described, but the biosignal measurement system of the present embodiment is applicable not only to the measurement of the electrocardiogram, but also to the measurement of other biosignals such as electromyograms and electroencephalogram measurement. By applying the biosignal measurement system of the present embodiment, it is possible to eliminate the discomfort of the wearer due to the physical wiring of the electrode devices, and it is possible to expect an effect of increasing the degree of freedom of electrode disposition and an increase in the range of gadgets to be mounted.

As the electrodes (21, 31) of the electrode devices (20, 30), electrodes of various materials and configurations can be used. Any electrode such as an Ag/AgCl electrode used in medical applications, a cloth electrode having conductivity, or a metal electrode can be used.

In addition, it is also possible to further improve the usability by using a non-contact electrode configuration in which an electrode is attached to the clothing by using an electrode made of cloth or metal that does not need to be directly attached to the body of the wearer.

Since the biopotential information is a very weak signal, signal amplification by the amplifier circuits (22, 32) using a filter circuit or an operational amplifier is required. In the amplifier circuits (22, 32) of the electrode devices (20, 30), a high input impedance is required in order to reduce a loss of the biopotential.

With an inverting amplifier circuit, the resistance for determining the input impedance also affects the gain setting, and further directly contributes as thermal noise, so that the SN ratio of the biopotential is lowered. On the other hand, a non-inverting amplifier circuit has a feature that noise is less likely to increase even in a high input impedance configuration. As the amplifier circuits (22, 32), it is effective to use a non-inverting amplifier circuit. By adopting the non-inverting amplifier circuit, it is possible to achieve a configuration equivalent to that of an instrumentation amplifier having a high capability of suppressing an in-phase component appearing as a noise component as a system.

Any wireless standard such as carrier communication, Wi-Fi (registered trademark), and Bluetooth (registered trademark) can be used as the wireless standard used in the wireless transmitters (24, 34) of the electrode devices (20, 30). It is sufficient if the biosignal generation device 40 that receives the information of the biopotential transmitted by the electrode devices (20, 30) is selected according to the communication standard to be used. When a short-range communication standard such as Bluetooth is used, a device carried by a wearer such as a smartphone can be used, and when a short-range communication standard such as Wi-Fi is used, a device such as a server can also be used.

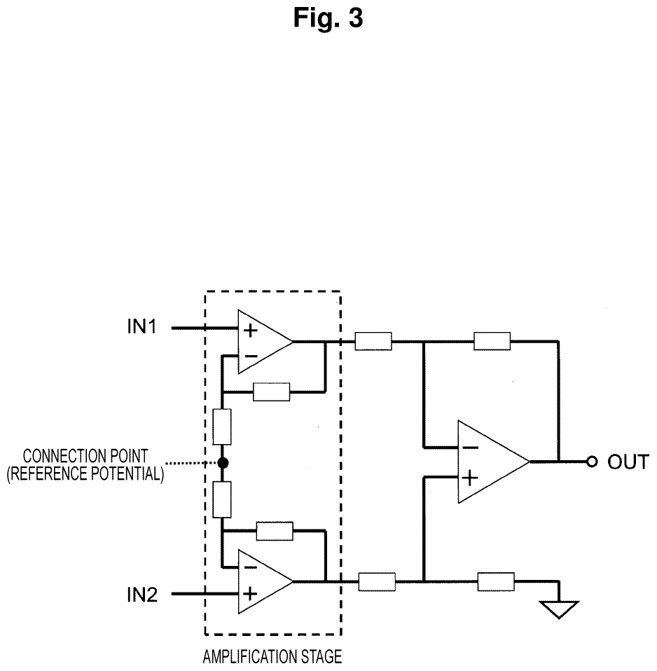

In the present embodiment, usability is improved by using the plurality of electrode devices (20, 30) without physical wiring. On the other hand, since the electrode devices (20, 30) are not connected by the physical wiring, there arises a problem that the reference potentials in the amplifier circuits of the electrode devices do not coincide with each other. As a measurement circuit generally used in a system in which conventional physical wiring exists, there is an instrumentation amplifier as illustrated in FIG. 3.

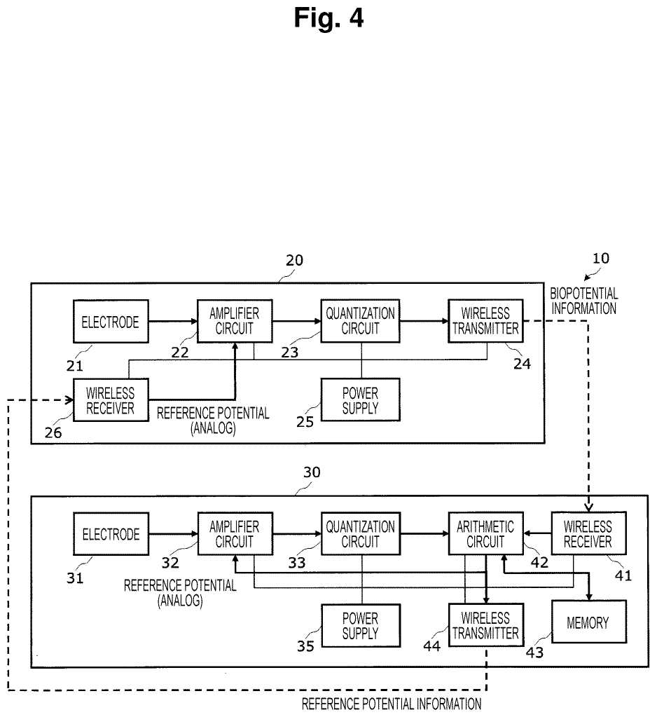

In biosignal measurement, in order to detect a potential difference between biopotentials measured by a plurality of electrodes, it is necessary to amplify a difference between two input potentials input to an input terminal of a differential amplifier circuit at a subsequent stage of the instrumentation amplifier of FIG. 4 while greatly suppressing a noise component of an in-phase component input to the input terminal. In order to suppress the noise component of the in-phase component, it is important that inverting input terminals of two non-inverting amplifier circuits at an amplification stage preceding the instrumentation amplifier of FIG. 3 are connected to each other, that is, the reference potentials of the two non-inverting amplifier circuits are common.

The potential at the connection point of the inverting input terminals of the two non-inverting amplifier circuits converges to an average value of the two input potentials and becomes a reference potential of the two non-inverting amplifier circuits. In FIG. 1, there is no physical wiring between the electrode devices (20, 30), and the inverting input terminals of the amplifier circuits (22, 32) of the electrode devices (20, 30) are not connected by the physical wiring. Therefore, the reference potentials of the amplifier circuits (22, 32) of the respective electrode devices (20, 30) do not coincide with each other, which may deteriorate the measurement accuracy.

In the present embodiment, in order to improve the degradation of the measurement accuracy due to the non-coincidence between the reference potentials, the reference potentials of the two divided non-inverting amplifier circuits are made common. Specifically, the reference potential generated by the biosignal generation device 40 is used as the reference potentials of the amplifier circuits (22, 32) in the electrode devices (20, 30), whereby the reference potentials are made common. As a result, signal amplification in which the reference potentials of the amplifier circuits (22, 32) are made common among the plurality of electrode devices (20, 30) becomes possible, the biosignal measurement accuracy is improved, and a good biosignal can be finally obtained.

The electrode devices (20, 30) of the present embodiment include the wireless receivers (26, 36) for receiving the reference potential information from the biosignal generation device 40. The amplifier circuits (22, 32) of the electrode devices (20, 30) amplify the biopotential using, as a reference potential, a potential obtained by converting the reference potential information received as a digital value into an analog value.

It is sufficient if a digital-analog converter (DAC) is used to convert a digital value into an analog value. In electronic equipment such as an audio, there is also a configuration in which a low-pass filter is provided at the output of the DAC, and the same configuration can suppress unstable oscillation of the amplifier circuit.

The arithmetic circuit 42 of the biosignal generation device 40 generates a reference potential when the biopotential is amplified in the electrode devices (20, 30) by using information of the biopotential measured by the plurality of electrode devices (20, 30). For example, in the arithmetic circuit 42 of the biosignal generation device 40, it is sufficient if the reference potentials are generated by using addition-averaging of the information of the biopotential measured by the plurality of electrode devices (20, 30).

Usually, since a communication module has both functions of transmission and reception, the wireless transmitters (24, 34) and the wireless receivers (26, 36) in FIG. 1 may be implemented by one communication module. The configuration is not limited to the configuration of FIG. 1, and other configurations may be used as long as it is possible to send the digitized biopotential information output from the quantization circuits (23, 33) to the biosignal generation device 40 and receive the reference potential information obtained by calculating the biopotential information from the biosignal generation device 40.

As an electrocardiogram that is one of biosignals, there is an electrocardiogram called a 12-lead electrocardiogram used for medical applications. As an electrode in the case of measuring the 12-lead electrocardiogram, ten electrodes are attached to the limbs and around the ribs, and a potential difference between a plurality of electrode pairs is measured. In the case of such an electrode disposition, since a large number of cables are entangled on the wearer's body and the wearer feels great discomfort, measurement other than in a lying position is not often performed.

By applying the biosignal measurement system of the present embodiment as a system for generating the 12-lead electrocardiogram, all of the above-described large number of cables can be removed. As a result, it is possible to eliminate discomfort to the wearer due to a large number of cables and to achieve 12-lead electrocardiogram measurement constantly in daily life, and it is also expected to contribute to medical progress.

### Second Embodiment

FIG. 4 is a diagram illustrating a configuration example of a biosignal measurement system according to a second embodiment of the present invention. A function required by a biosignal generation device is to receive information of biopotentials transmitted from a plurality of electrode devices and generate a biosignal and reference potential information by using the received information of biopotentials. As in the configuration example of FIG. 4, the function of a biosignal generation device 40 may be implemented in any of electrode devices 20, 30.

In the following description, the electrode device 30 in which the function of the biosignal generation device 40 is implemented is referred to as a master unit, and the electrode device 20 that transmits a signal of the measurement potential to the master unit is referred to as a slave unit, and the operation of the present embodiment will be described.

A wireless receiver 41 of the master unit receives the information of the biopotential measured by the slave unit, and an arithmetic circuit 42 of the master unit generates a biosignal and reference potential information by using the information of the biopotential measured by the master unit and the information of the biopotential measured by the slave unit. The generated biosignal is stored in a memory 43 of the master unit, can be used when the biosignal is analyzed, and can achieve a function similar to that of the biosignal generation device 40 of the first embodiment.

The reference potential information generated in the master unit is used as a reference potential in an amplifier circuit 32 of the master unit, and is transmitted to the slave unit via a wireless transmitter 44. An amplifier circuit 22 of the slave unit amplifies the biopotential using the reference potential obtained by converting the reference potential information received from the master unit into analog data. In the present embodiment, since the biosignal generation device 40 is not necessary as a device apart from the electrode devices, it is not necessary to carry a device such as a smartphone, and it is possible to achieve biosignal measurement with less limitation on the user.

### Third Embodiment

According to the first embodiment and the second embodiment, it is possible to measure a biosignal with accuracy equivalent to that of a conventional biosignal measurement system using a plurality of electrode devices without physical wiring. On the other hand, there is a problem that a delay occurs when the biopotential information and the reference potential information are transmitted between the biosignal generation device 40 and the electrode devices (20, 30) by wireless communication including digital logic. The delay amount varies depending on the communication protocol, but is, for example, about 10 msec in Bluetooth.

In a conventional biosignal measurement system with physical wiring, the delay can be regarded as almost zero. On the other hand, in the present embodiment, in a case where the delay amount due to wireless transmission is large, there is a case where the effect cannot be negligible. Since the reference potential is generated by adding two pieces of biopotential information, a differential mode component that is a signal component is suppressed, and only a common mode (in-phase) component that is a noise component is obtained.

An output voltage Vo when the amplifier circuit of the electrode device is a non-inverting amplifier circuit is indicated by Formula (1) described below. S(t) and C(t) are a signal component and a common mode component, respectively, and have periodicity.

$$[ Math . 1 ]$$

$$V o = K ( Vin - V c ) + V c = K ( S ( t ) + C ( t ) - C ( t - ϕ ) ) + C ( t - ϕ ) \qquad ( 1 )$$

$$K : Amplification factor , ϕ : Delay amount ,$$

$$Vin : Input voltage , V c : Reference potential$$

According to Formula (1) above, in a case where there is a delay amount in which the phase of the common mode component is inverted, the common mode component is not suppressed, but rather emphasized. As a result, saturation or oscillation of the output occurs in the amplifier circuit, and there is a possibility that the biopotential cannot be measured.

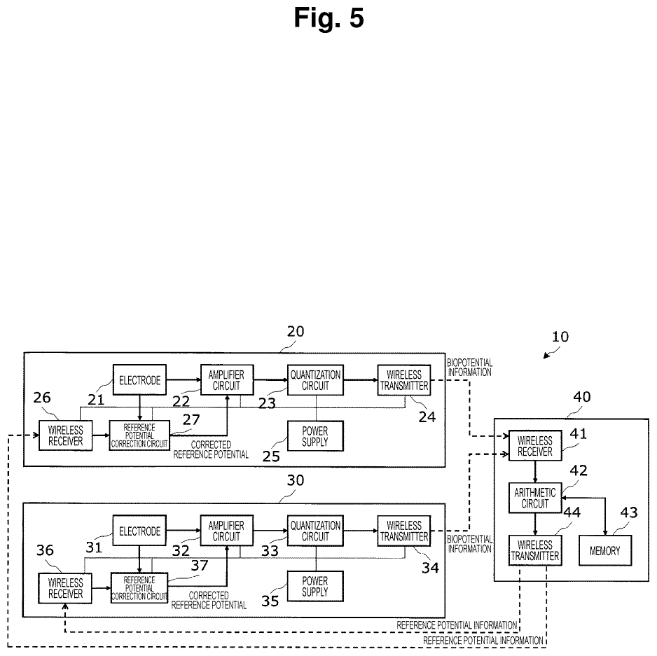

In order to solve this problem, in the third embodiment, the phase variation of the reference potential information caused by the propagation delay is corrected using stored time-series data of the reference potential information. FIG. 5 is a diagram illustrating a configuration example of a biosignal measurement system according to a third embodiment of the present invention. In the present embodiment, as illustrated in FIG. 5, reference potential correction circuits (27, 37) are provided in electrode devices (20, 30), a certain amount of time-series data of received reference potential information is stored, and a correlation value of the stored time-series data of the reference potential information is calculated, thereby estimating a lag in the time-series data of the reference potential information, and correcting a phase variation of the reference potential information on the basis of the estimated lag.

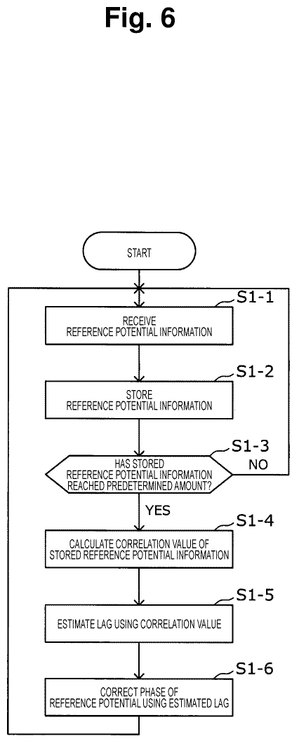

FIG. 6 is a diagram illustrating an example of an operation flow in a reference potential correction circuit according to the third embodiment of the present invention. In the present embodiment, the time-series data of a predetermined amount of reference potential information is stored (S1-1 to S1-3), the correlation value of the stored data of the reference potential information is calculated (S1-4), the lag that is the delay amount in the time-series data of the reference potential information is estimated using the calculated correlation value (S1-5), and the phase variation of the reference potential information is corrected according to the value of the estimated lag (S1-6). By correcting the phase of the reference potential information according to the value of the lag estimated using the correlation value, it is possible to correct the phase variation caused by the propagation delay and to measure the biopotential while suppressing saturation and oscillation. Note that it is sufficient if a ring buffer is used to store the time-series data of the reference potential information. The use of the ring buffer enables efficient memory arrangement.

### Fourth Embodiment

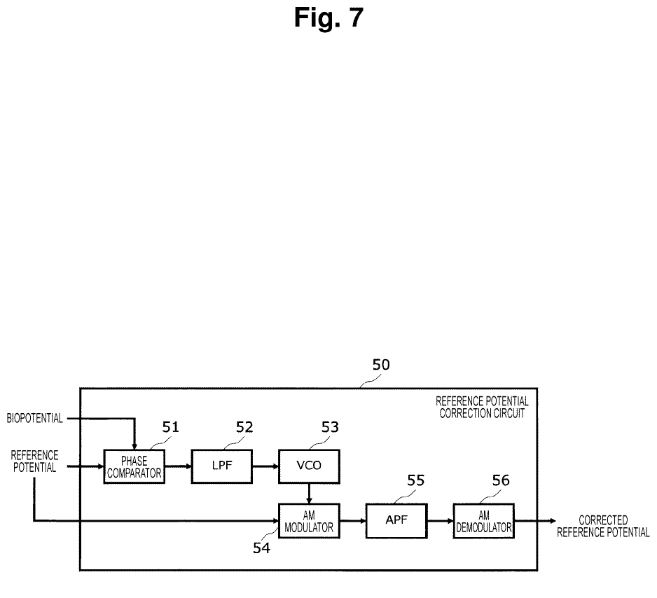

FIG. 7 is a diagram illustrating a configuration example of a reference potential correction circuit according to a fourth embodiment of the present invention. A reference potential correction circuit 50 of the present embodiment converts reference potential information received from a biosignal generation device into an analog signal, then performs phase-comparison with a signal of biopotential input from an electrode portion, and corrects the phase of the reference potential according to the obtained phase difference. By correcting the phase in the analog region, it is not necessary to perform digital calculation as in the third embodiment, so that power consumption can be greatly suppressed.

As illustrated in FIG. 7, for example, the reference potential correction circuit 50 of the present embodiment can be achieved by a phase comparator 51, a low-pass filter (LPF) 52, a voltage-controlled oscillator (VCO) 53, an AM modulator 54, an all-pass filter (APF) 55, and an AM demodulator 56.

In the present embodiment, a signal having a frequency corresponding to the phase difference between the biopotential signal and the reference potential signal is output by using the phase comparator 51 that compares the phases of the biopotential signal and the reference potential signal, the LPF 52 that outputs a direct-current component from the output of the phase comparator 51, and the VCO 53 that is an oscillator that outputs a signal having a frequency corresponding to the output of the LPF 52. In the AM modulator 54, the reference potential signal is amplitude-modulated by the output signal of the VCO 53, and the amplitude-modulated reference potential signal is caused to pass through the APF 55.

Since the APF 55 changes only the phase without changing the amplitude of the reference potential signal, it is possible to shift the phase of the reference potential signal according to the frequency of the output signal of the VCO 53, that is, the phase difference detected by the phase comparator 51 by causing the reference potential signal that has been amplitude-modulated using the output signal of the VCO 53 to pass through the APF 55. By demodulating the output signal of the all-pass filter with the AM demodulator 56, a reference potential signal whose phase is shifted can be obtained.

### Fifth Embodiment

According to the first embodiment and the second embodiment, it is possible to measure a biosignal with accuracy equivalent to that of a conventional biosignal measurement system using a plurality of electrode devices without physical wiring. On the other hand, since the output of the reference potential changes stepwise depending on the sampling rate of the device, the reference potential becomes constant until a next sampling value is transmitted, and the signal of the reference potential changes stepwise. However, since the true reference potential always changes, this difference is output as an error, and there is a problem that the suppression performance of the common mode (in-phase) component is lowered.

In order to solve the above problem, in a fifth embodiment, reference potential correction circuits provided in electrode devices (20, 30) interpolate a signal of a reference potential that changes stepwise. Since the signal of the reference potential in amplifier circuits (22, 32) include a periodic noise component called a hum of 50 or 60 Hz, it is possible to interpolate the signal of the reference potential that changes stepwise according to the sampling rate of an electrode device by estimating the periodic signal. The interpolation of the signal of the reference potential can be implemented in both the electrode devices (20, 30) and a biosignal generation device 40.

In the case of implementation in the electrode devices (20, 30), this is achieved by interpolating a reference potential until next reception after receiving a signal of a reference potential. On the other hand, in the case of implementation in the biosignal generation device 40, it is sufficient if a result of interpolation in the biosignal generation device 40 is transmitted to the electrode devices (20, 30). This interpolation may be performed by specifying frequency information by Fourier transform or wavelet transform.

In addition, in a case where the above-described interpolation is performed, interpolation can be performed with a small amount of calculation by using an auto-regressive (AR) model, and there is an advantage that the interpolation can be easily implemented even in the electrode devices (20, 30). The AR model approximates a signal using a plurality of coefficients called AR coefficients. The number of AR coefficients is a parameter that can be arbitrarily determined by a designer, but the amount of data is generally smaller than that of data used for estimation. Therefore, by obtaining the AR coefficients in the biosignal generation device 40 and sending this value to the electrode devices (20, 30), the communication amount can be reduced, the calculation amount in the electrode devices (20, 30) can be reduced, and the power consumption of the electrode devices (20, 30) can be expected to be reduced.

### INDUSTRIAL APPLICABILITY

The embodiments of the present invention can be used in a bioelectrode used for acquiring a biosignal such as an electrocardiographic signal on a daily basis and a biosignal measurement system using the bioelectrode.

Reference Signs List

| | |
|---|---|
| 1 | Wearer |
| 2 | Clothing |
| 10 | BIOSIGNAL MEASUREMENT SYSTEM |
| 20, 30 | Electrode device |
| 21, 31 | Electrode |
| 22, 32 | Amplifier circuit |
| 23, 33 | Quantization circuit |
| 24, 34 | Wireless transmitter |
| 25, 35 | Power supply |
| 26, 36 | Wireless receiver |
| 40 | Biosignal generation device |
| 41 | Wireless receiver |
| 42 | Arithmetic circuit |
| 43 | Memory |

1-8. (canceled)

9. A biosignal measurement system comprising:
a plurality of electrode devices, each including an electrode, an amplifier circuit configured to amplify a biopotential measured via the electrode, a quantization circuit configured to convert the biopotential amplified by the amplifier into digital data to generate biopotential information, a transmitter configured to transmit the biopotential information, and a receiver configured to receive reference potential information of the amplifier; and
a biosignal generation device including a wireless receiver configured to receive the biopotential information transmitted from the plurality of electrode devices, an arithmetic circuit configured to generate a biosignal waveform and the reference potential information using the biopotential information in at least two of the plurality of electrode devices, and a transmitter configured to transmit the reference potential information to the plurality of electrode devices, wherein
the amplifier of each of the plurality of electrode devices is configured to amplify the biopotential using reference potential information received from the biosignal generation device.

10. The biosignal measurement system according to claim 9, wherein
the plurality of electrode devices include a master electrode device which is one of the plurality of electrode devices and a slave electrode device which is the electrode device other than the master electrode device,
the biosignal generation device is implemented in the master electrode device, wherein
the arithmetic circuit implemented in the master electrode device is configured to generate the biosignal waveform and the reference potential information using the biopotential information obtained in the master electrode device and the biopotential information obtained in the slave electrode device.

11. The biosignal measurement system according to claim 9, wherein each of the plurality of electrode devices includes a reference potential correction circuit configured to correct a phase variation of the reference potential information caused by a propagation delay with respect to the biosignal generation device, wherein the amplifier is configured to amplify the biopotential using the reference potential information corrected in the reference potential correction circuit.

12. The biosignal measurement system according to claim 11, wherein the reference potential correction circuit is configured to estimate a lag in data of the reference potential information using a correlation value calculated using data of a plurality of pieces of the reference potential information, and is configured to correct the phase variation of the reference potential information on a basis of the estimated lag.

13. The biosignal measurement system according to claim 11, wherein
the reference potential correction circuit includes a phase comparator configured to perform phase-comparison between a signal of the biopotential and a signal of a reference potential obtained from the reference potential information, a low-pass filter configured to output a direct-current component of an output of the phase comparator, an oscillator configured to output a signal of a frequency corresponding to an output of the low-pass filter, a modulator configured to amplitude-modulate the reference potential information using an output of the oscillator, an all-pass filter configured to input an output signal of the modulator, and a demodulator configured to demodulate an output signal of the all-pass filter as the corrected reference potential.

14. The biosignal measurement system according to claim 9, wherein each of the plurality of electrode devices further includes a reference potential correction circuit configured to interpolate a reference potential changing stepwise according to a sampling rate of the electrode device by estimating a periodic signal included in a signal of the reference potential in the amplifier circuit, wherein the amplifier is configured to amplify the biopotential using the reference potential interpolated in the reference potential correction circuit.

15. The biosignal measurement system according to claim 14, wherein the reference potential correction circuit is configured to interpolate the reference potential using an AR model.

16. The biosignal measurement system according to claim 15, wherein the reference potential correction circuit is configured to interpolate the reference potential information changing stepwise using an AR coefficient calculated by the biosignal generation device.

17. The biosignal measurement system according to claim 10, wherein each of the plurality of electrode devices includes a reference potential correction circuit configured to correct a phase variation of the reference potential information caused by a propagation delay with respect to the biosignal generation device, wherein the amplifier is configured to amplify the biopotential using the reference potential information corrected in the reference potential correction circuit.

18. The biosignal measurement system according to claim 10, wherein each of the plurality of electrode devices further includes a reference potential correction circuit configured to interpolate a reference potential changing stepwise according to a sampling rate of the electrode device by estimating a periodic signal included in a signal of the reference potential in the amplifier circuit, wherein the amplifier is configured to amplify the biopotential using the reference potential interpolated in the reference potential correction circuit.

19. An electrode device comprising:
an electrode;
an amplifier circuit configured to amplify a biopotential measured via the electrode;
a quantization circuit configured to convert the biopotential amplified by the amplifier into digital data to generate biopotential information;
a transmitter configured to transmit the biopotential information, and a receiver configured to receive reference potential information of the amplifier;
a wireless receiver configured to receive the biopotential information transmitted from another electrode device;
an arithmetic circuit configured to generate a biosignal waveform and the reference potential information using the biopotential information in at least two of the plurality of electrode devices; and
a transmitter configured to transmit the reference potential information to the plurality of electrode devices; wherein
the amplifier is configured to amplify the biopotential using the reference potential information.

20. The electrode device according to claim 19, further comprising:
a reference potential correction circuit configured to correct a phase variation of the reference potential information caused by a propagation delay with respect to the biosignal generation device, wherein the amplifier is configured to amplify the biopotential using the reference potential information corrected in the reference potential correction circuit.

21. The electrode device according to claim 20, wherein
the reference potential correction circuit estimates a lag in data of the reference potential information using a correlation value calculated using data of a plurality of pieces of the reference potential information, and corrects the phase variation of the reference potential information on a basis of the estimated lag.

22. The electrode device according to claim 20, wherein the reference potential correction circuit includes:
a phase comparator configured to perform phase-comparison between a signal of the biopotential and a signal of a reference potential obtained from the reference potential information;
a low-pass filter configured to output a direct-current component of an output of the phase comparator;
an oscillator configured to output a signal of a frequency corresponding to an output of the low-pass filter;
a modulator configured to amplitude-modulate the reference potential information using an output of the oscillator;
an all-pass filter configured to input an output signal of the modulator; and
a demodulator configured to demodulate an output signal of the all-pass filter as the corrected reference potential.

23. The electrode device according to claim 19, further comprising:
a reference potential correction circuit configured to interpolate a reference potential changing stepwise according to a sampling rate of the electrode device by estimating a periodic signal included in a signal of the reference potential in the amplifier circuit, wherein the amplifier is configured to amplify the biopotential using the reference potential interpolated in the reference potential correction circuit.

24. The electrode device according to claim 23, wherein the reference potential correction circuit is configured to interpolate the reference potential using an AR model.

25. The electrode device according to claim 24, wherein the reference potential correction circuit is configured to interpolate the reference potential information changing stepwise using an AR coefficient calculated by the biosignal generation device.

26. A method of measuring a biosignal, the method comprising:
measuring a biopotential via electrodes of a plurality of electrode devices;
amplifying the measured biopotential in each of the plurality of electrode devices using an amplifier circuit;
converting the amplified biopotential into digital data to generate biopotential information using a quantization circuit in each of the plurality of electrode devices;
transmitting the biopotential information from each of the plurality of electrode devices to a biosignal generation device;
generating, by the biosignal generation device, a biosignal waveform and reference potential information using the biopotential information from at least two of the plurality of electrode devices;
transmitting the reference potential information from the biosignal generation device to the plurality of electrode devices; and
amplifying the biopotential in each of the plurality of electrode devices using the reference potential information received from the biosignal generation device.

27. The method according to claim 26, further comprising:
implementing the biosignal generation device in one of the plurality of electrode devices as a master electrode device; and
generating the biosignal waveform and the reference potential information using biopotential information obtained in the master electrode device and biopotential information obtained in at least one other electrode device.

28. The method according to claim 26, further comprising:
correcting, in each of the plurality of electrode devices, a phase variation of the reference potential information caused by a propagation delay with respect to the biosignal generation device; and
amplifying the biopotential using the corrected reference potential information.
